(19) United States
(12) Patent Application Publication
(10) Pub. No.: US 2009/0143769 A1
(43) Pub. Date: Jun. 4, 2009

(54) Guidewire with adjustable stiffness

(75) Inventors: Juan Carlos Parodi, Pinecrest, FL (US); Peter W.J. Hinchliffe, Campbell Hall, NY (US); Henry Lupton, Oranmore (IE); Ivan Mooney, Tuam (IE); Colin Donohue, Athenry (IE)

Correspondence Address: Neil D. Gershon, 1011 High Ridge Road, Stamford, CT, 06905, US

(73) Assignee: Interventional & Surgical Innovations, LLC

(21) Appl. No.: 12/291,851

(22) Filed: Nov. 14, 2008

Related U.S. Application Data
(63) Continuation of application No. 12/082,507, filed on Apr. 11, 2008.
(60) Provisional application No. 60/913,489, filed on Apr. 23, 2007.
(60) Provisional application No. 61/008,100, filed on Dec. 17, 2007.

(51) Int. Cl. A61M 25/09

(52) U.S. Cl. 604/528

(57) ABSTRACT

A medical guidewire system comprising a first inner member having a first outer diameter, a second intermediate member having a second outer diameter larger than the first outer diameter, and a third outer member having a third diameter larger than the second outer diameter of the second member. The second member has a longitudinal extending opening to receive the first member for sliding movement with respect to the first member and the third outer member has a longitudinally extending opening to receive the second member for sliding movement with respect to the first and second member. The first member has a first stiffness, the third member has a third stiffness greater than the first stiffness, and the second member is movable to stiffen the guidewire system.

### BACKGROUND OF THE INVENTION

This application claims priority from provisional application Ser. No. 60/913,489, filed Apr. 23, 2007 and provisional application Ser. No. 61/008,100, filed Dec. 17, 2007. The entire contents of each of these applications is incorporated herein by reference.

1. Technical Field

This application relates to a medical guidewire and more particularly to a medical guidewire system with adjustable size and stiffness.

2. Background of Related Art

Guidewires are currently being used in medical procedures to guide catheters, sheaths or other devices from a remote site to a surgical site. From a remote part of the body, a guidewire is introduced into an artery or vein. The guidewire is then advanced through the vascular system to the target site where an angiogram, balloon, stent, catheter or other vascular device is to be positioned. The guidewire then functions as a rail for advancement of these devices.

Currently, a soft small diameter wire, such as a 0.014 wire, is utilized initially to advance in the artery or vein. During advancement, especially through tortuous anatomy, the soft wire may lack the requisite pushability to advance around a curve. Also, due to its softness/flexibility, it may be difficult to advance a catheter over it to perform the surgical, e.g. diagnostic and/or interventional, procedure. In these instances, this flexible wire needs to be exchanged for a stiffer and/or larger wire. To exchange the guidewire, several steps are required. First, an exchange catheter is advanced over the soft wire. Second, the soft wire is removed. Third, the stiffer wire is inserted through the exchange catheter. Fourth, the exchange catheter is removed, leaving the stiffer wire in place. Such wire exchanges are time consuming and require two separate wires and an exchange catheter. Furthermore, these steps also increase risks to the patient such as increased risk of infection and increased chance of damaging the vessel due to the added insertion and removal of the wires through the vascular system as well as possible loss of wire position and critical time loss.

Even after exchange for the larger wire, sometimes the requisite stiffness and pushability to advance through a curved vessel portion is still lacking and therefore the wire needs to be exchanged for yet an even stiffer wire. This requires an additional wire exchange utilizing the time consuming four step method described above.

After such exchange for a stiffer wire and advancement around the tortuous portion of the anatomy, a stenosis or restricted passage of the vessel might be encountered through which the larger wire cannot pass. Thus, yet another catheter exchange could be required, this time exchanging the larger diameter stiffer wire for the smaller diameter softer wire. As a result, multiple guidewire exchanges requiring multiple insertions of the exchange catheter, multiple removals of the already inserted wire, and multiple insertions of a new wire from the remote site may be necessary in a single surgical (diagnostic and/or interventional) procedure. As noted above, this adds undesired time to the surgical procedure, as well as increases the risk of trauma or damage to the vessel and loss of desired wire position.

In addition, the inventor has found that in some instances where a catheter exchange is required, the surgical procedure cannot even be performed. That is, in some instances, the exchange catheter, which has a larger diameter (typically about 0.040 inches inside diameter) than the stiffer replacement wire because it has a lumen to receive the wire, cannot cross the stenosis. In this case, the guidewire with increased pushability cannot be inserted and advanced to reach the target site, thus not enabling a stent, dilation balloon or other vascular treatment device to be advanced to the surgical site. Consequently, the intralumenal surgical procedure cannot be performed.

As can be appreciated from the above, in the current procedure, multiple guidewires may be required to achieve desired parameters such as softness to reduce trauma to the vessel during insertion, reduced diameter to enable access through restricted passages in the vessels and facilitate access to the surgical site, stiffness/rigidity to allow pushability and stiffness/rigidity to facilitate passage of a catheter thereover. For example, a gentler more flexible guidewire, such as a 0.014 inch diameter wire, has the small diameter and softness advantage, but lacks the pushability to advance through some tortuous anatomy. The larger diameter guidewire, such as the 0.035 or 0.038 inch diameter guidewire, is more rigid and has better pushability but may be too large for restricted passages. It may also still lack the necessary stiffness, thus requiring an exchange for an extra stiff wire. The extra stiff wire lacks the flexibility and softness. Thus, the user needs to exchange the wires to obtain the requisite pushability, flexibility and stiffness for accessing the diagnostic and/or interventional site.

Also, exchange sheaths, when used with a 0.014 guidewire, present a relatively large stepped transition from their distal end to the smaller diameter 0.014 guidewire, therefore creating a more traumatic “snow plow” effect during insertion.

Therefore, it would be advantageous to provide a guidewire system which provides the desired diameter, pushability, flexibility and stiffness without requiring guidewire exchanges and exchange catheters, thereby eliminating the foregoing disadvantages of such exchanges.

### SUMMARY OF THE INVENTION

The present invention overcomes the problems and deficiencies of the prior art. The present invention provides a medical guidewire system comprising a first inner member having a first outer diameter, a second intermediate member having a second outer diameter larger than the first outer diameter, and a third outer member having a third diameter larger than the second outer diameter. The second member has a longitudinally extending opening to receive the first member for relative sliding movement with respect to the first member and the third outer member has a longitudinally extending opening to receive the second member for relative sliding movement with respect to the first and second member. The first member has a first stiffness, the third member has a third stiffness greater than the first stiffness, and the second member is movable with respect to the third member to provide the third member with a second stiffness greater than the third stiffness.

In one embodiment, the first member comprises a solid core material. The first and second members in one embodiment are composed at least in part of shape memory metal. In one embodiment, the second and/or third members comprise hypotubes which can have slots in a sidewall to increase flexibility.

In one embodiment one or more of the members has a handle at the proximal end. The handle attached to the first inner member can be removable to enable removal of the second and third members from the guidewire system. In one embodiment, the handle of the first member interlocks with the handle of the second member to fix the position of the first and second members with respect to each other. The handles can interlock by various structures including for example a pin and slot, mating tabs, male/female tapers providing an interference fit, and a compressible clamping member.

In one embodiment, the first member can have an enlarged distal tip exceeding the inner diameter of the third member, or at least exceeding a diameter of the opening to the lumen of the third member, to prevent full withdrawal of the distal tip of the first member into the lumen of the third member.

In one embodiment, a stop is provided to limit relative movement of the second and third members such that a distalmost end of the second member cannot extend to a distalmost end of the third member, thus ensuring some degree of flexibility at the distalmost end of the guidewire system.

The present invention also provides a multi-component medical guidewire system comprising first, second and third coaxially positioned members relatively slidable with respect to one another, wherein the second member is coaxially positioned between the first and third members and has a sufficient stiffness to selectively increase the stiffness of the guidewire system upon positioning within a distal portion of the third member. In one embodiment, the second member interlocks with the third member and/or the first member to fix the respective members in position.

In a preferred embodiment, the first member has a diameter of about 0.014 inches and the third member has a diameter of about 0.035 to about 0.038 inches.

The present invention also provides a multi-component guidewire system comprising first, second and third coaxially positioned members relatively slidable with respect to one another with each of the members having an engagement region at the proximal end portion. The engagement region has an interlocking feature to interlock with another engagement region to fix the relative position of the respective members. In one embodiment, the engagement region of the first member is formed on a removable handle. In one embodiment, the interlocking feature comprises a tapered region on the handle which engages a mating region of another handle.

The present invention also provides a method of adjusting the stiffness and size of a guidewire without full withdrawal of the guidewire from a patient's vascular system, the method comprising:

a) providing a guidewire system comprising an inner member having a first outer diameter and a first stiffness, an outer member having a third larger diameter and a third stiffness, and a stiffener positioned between the inner and outer members and having a second outer diameter larger than the first diameter and smaller than the third diameter;

b) advancing the guidewire system into the vascular system with the outer member and stiffener in the retracted position to expose a substantial length of the inner member to expose a smaller member diameter;

c) after advancement of the guidewire system through the vascular system in step (b), changing the relative position of the outer member and inner member to provide a stiffer member to increase the pushability of the guidewire system if desired; and

d) thereafter, if desired, selectively advancing the stiffener to further increase the stiffness of the guidewire system to a second stiffness greater than the third stiffness.

The method can also include the step of detaching a proximal handle of the inner member to enable complete removal of the outer member and stiffener to leave the inner member in position for over the wire catheter or device insertion. An extension wire can optionally be attached to the proximal end of the inner member.

The present invention also provides a method of adjusting the stiffness of a guidewire extending into the vascular system of a patient, the method comprising:

a) providing a guidewire having an inner member having a first outer diameter and a first stiffness, an outer member having a third larger diameter and a third stiffness, and a stiffener positioned between the inner and outer members and having a second outer diameter larger than the first diameter and smaller than the third diameter;

b) advancing the guidewire into the vascular system from a remote site with the outer member and stiffener in the retracted position to expose a substantial length of the inner member;

c) when encountering a tortuous vessel portion wherein the inner member lacks the requisite pushability, changing the relative positions of the inner member and the outer member without removing the inner member from the patient so the outer member covers a distal portion of the inner member to create a stiffer guidewire; and

d) when encountering a restricted passage in a portion of the vessel, changing the relative positions of the inner member and outer member to expose at least a portion of the covered distal portion of the inner member.

The method can further comprise the step of advancing the stiffener over the inner member to increase the stiffness of the guidewire when encountering a tortuous passage of the vessel in which the outer member lacks the requisite pushability.

The present invention also provides a method of adjusting the stiffness of a guidewire extending into the vascular system of a patient, the method comprising:

a) providing a guidewire having an inner member having a first outer diameter and a first stiffness, an outer member having a third larger diameter and a third stiffness, and a stiffener positioned between the inner and outer members and having a second outer diameter larger than the first diameter and smaller than the third diameter;

b) advancing the guidewire into the vascular system from a remote site with the outer member in the extended position and the stiffener and inner member in an unexposed retracted position;

c) when encountering a vessel portion wherein the outer member is too large for advancement or lacks the requisite flexibility, changing the relative positions of the inner member and the outer member without removing the outer member from the patient so a portion of the inner member is exposed; and

d) when encountering a tortuous vessel portion wherein the inner member lacks the requisite pushability, changing the relative positions of the inner member and outer member to retract the inner member to leave the outer member as the distalmost region of the guidewire.

The foregoing methods, in one embodiment can include the step of interlocking the stiffener and the outer member to fix the position of the stiffener and the outer member and/or the step of interlocking the stiffener and the inner member to fix the position of the inner member.

### DETAILED DESCRIPTION OF THE DRAWINGS

Preferred embodiment(s) of the present disclosure are described herein with reference to the drawings wherein:

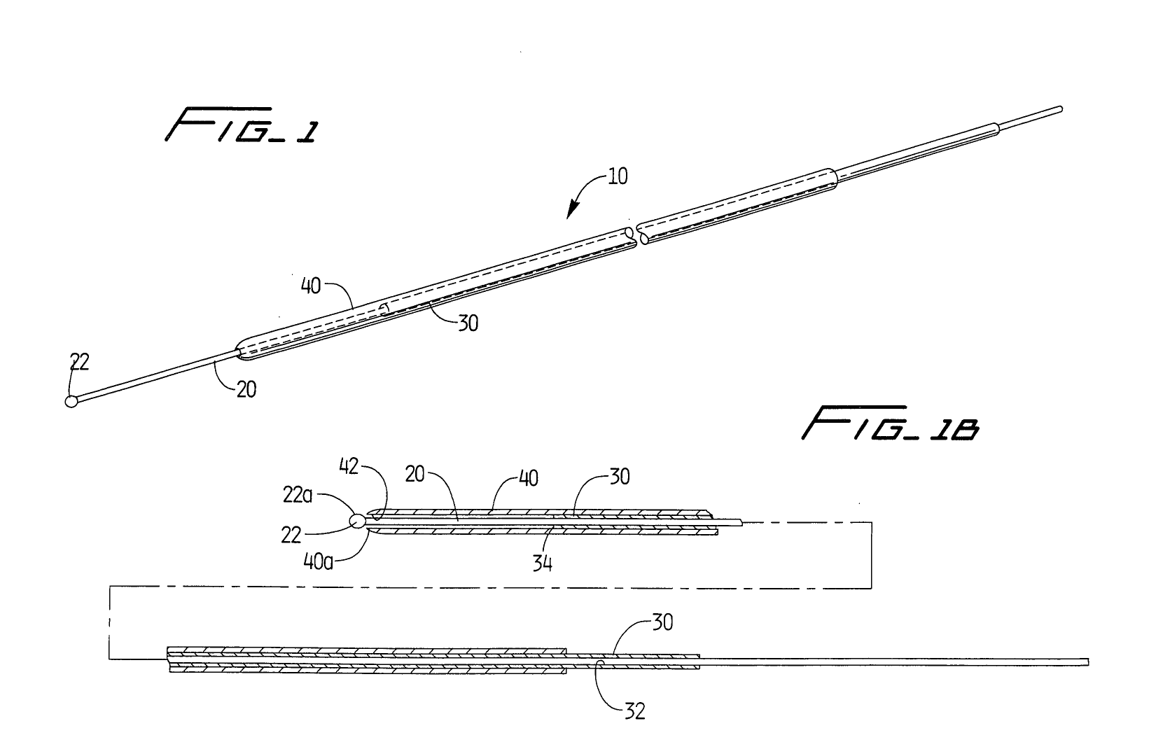

FIG. 1 is a perspective view of the guidewire system of the present invention showing the intermediate (stiffener) wire and outer wire in the retracted position to expose the inner wire;

FIG. 1A is an exploded perspective view of the guidewire of FIG. 1;

FIG. 1B is a longitudinal cross-sectional view of the guidewire of FIG. 1 showing the outer wire and the intermediate stiffener wire in the advanced position;

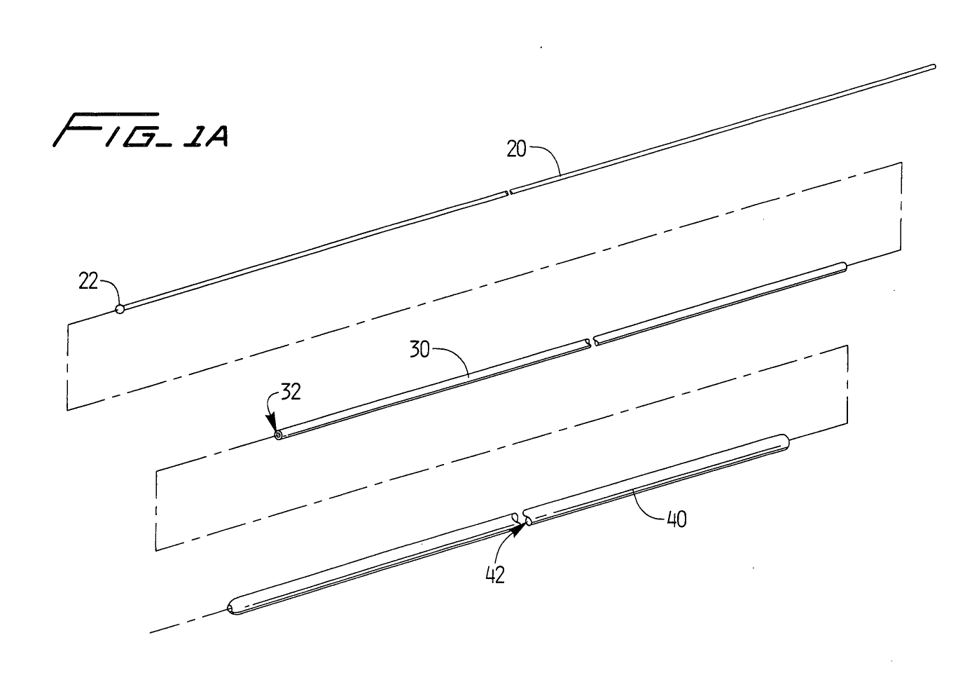

FIG. 2 is a perspective view of an alternate embodiment of the guidewire system of the present invention showing the intermediate (stiffener) wire and outer wire in the retracted position to expose the inner wire;

FIG. 2A is a longitudinal cross-sectional view of the guidewire of FIG. 2 showing the outer wire and the intermediate stiffener wire in the advanced position;

FIG. 3 is an anatomical view illustrating the guidewire of the present invention being inserted through the femoral artery for subsequent advancement through the vascular system, e.g. to the external carotid artery (the shuttle sheath not shown for clarity);

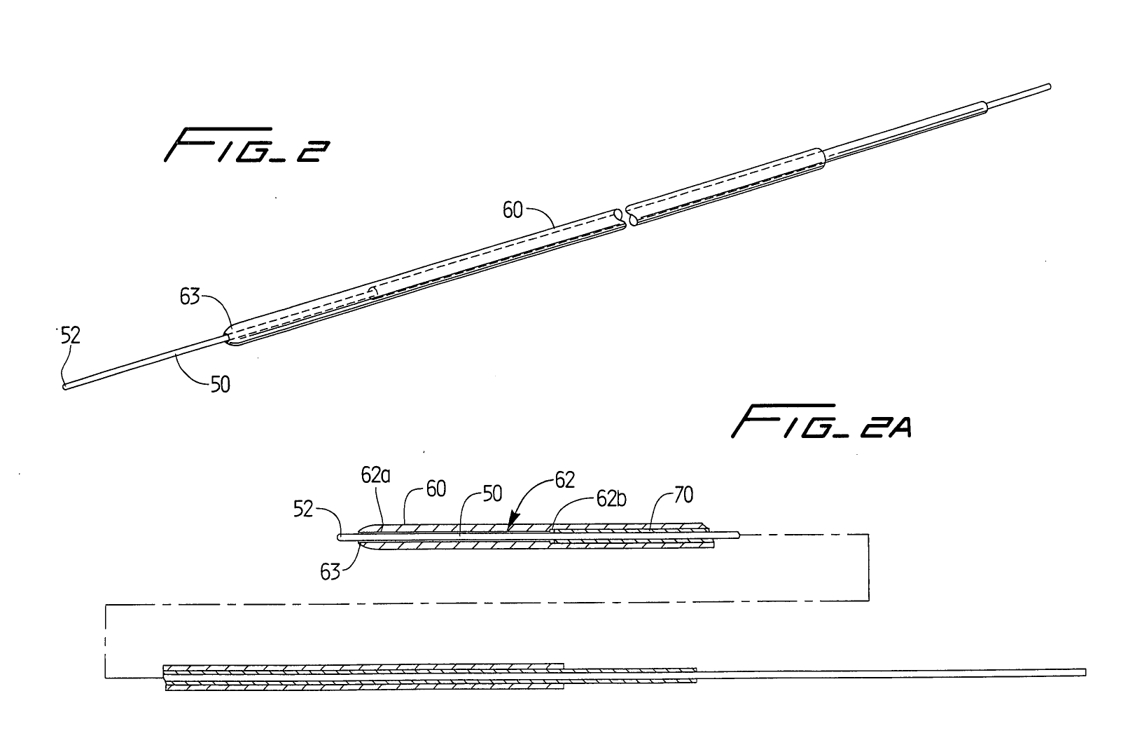

FIG. 4 is a longitudinal cross-sectional view of the guidewire of FIG. 1 showing the outer wire and the intermediate stiffener wire in the retracted position to expose the inner wire, corresponding to the position of the wires in FIG. 1;

FIG. 5 is a longitudinal cross-sectional view of the guidewire of FIG. 1 showing the outer wire in the advanced position and the intermediate stiffener wire in the retracted position;

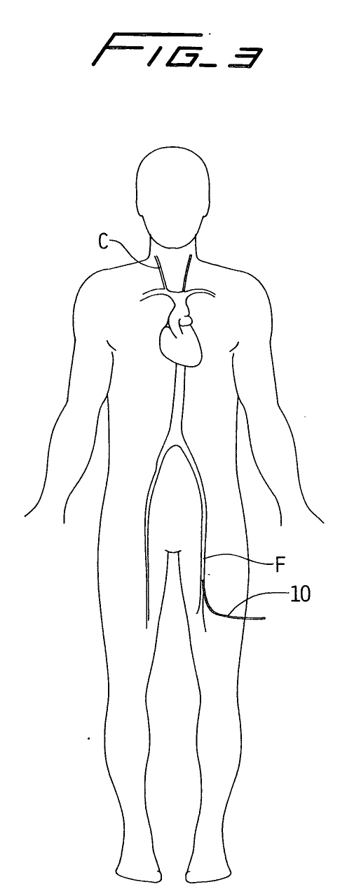

FIG. 6 is a perspective view of an alternate embodiment of the guidewire of the present invention having a modified distal tip, and illustrating the outer wire and intermediate stiffener wire in the retracted position to expose the inner wire;

FIG. 7 is a longitudinal cross-sectional view of the guidewire of FIG. 6 except showing the outer wire in the advanced position and the intermediate wire in the retracted position;

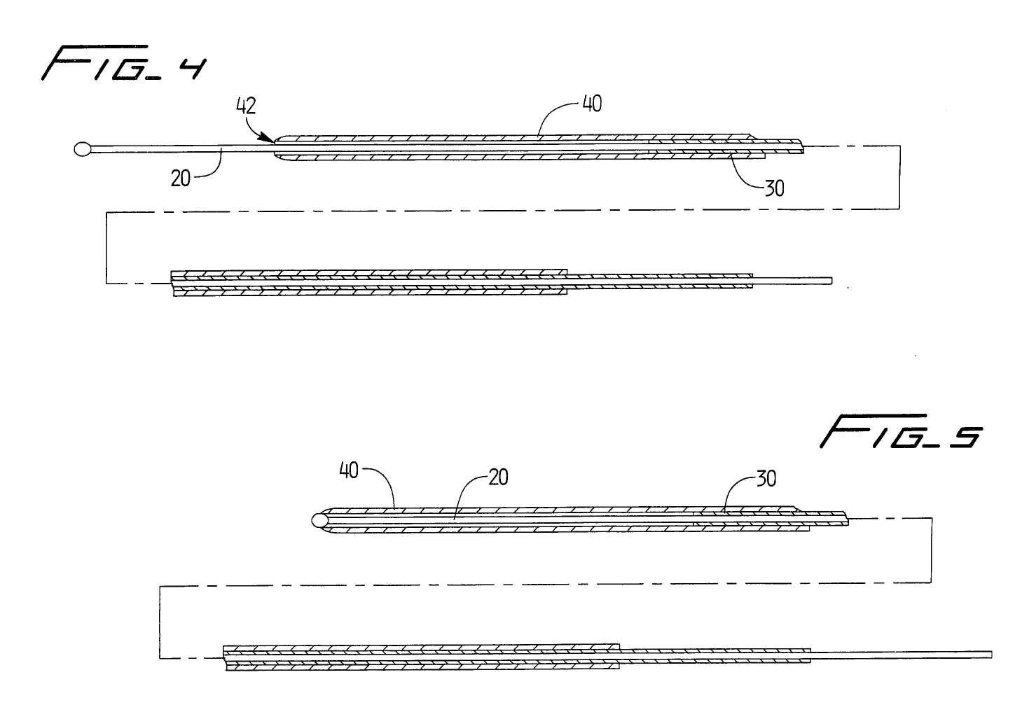

FIG. 8 is a perspective view of a proximal end of the guidewire of the present invention showing attachment of a conventional extension wire to the inner wire;

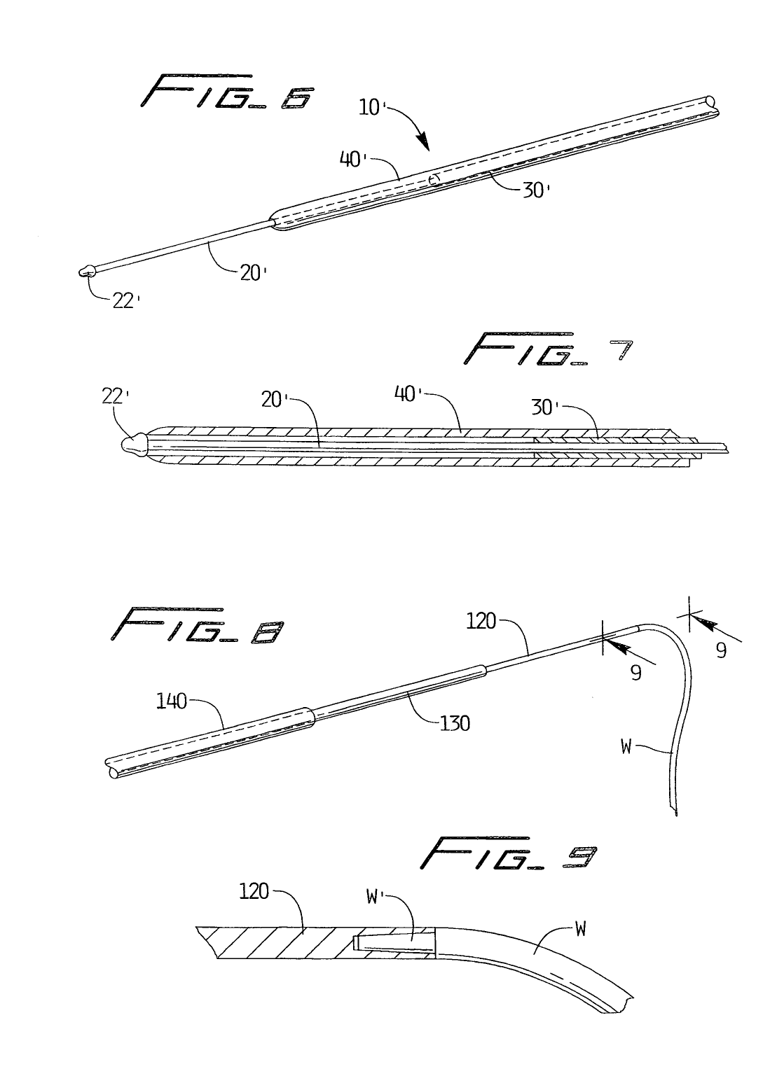

FIG. 9 is an enlarged cross-sectional view taken along line 9-9 of FIG. 8 showing the attachment of the extension wire to the inner wire;

FIG. 10 is a perspective view of another alternate embodiment of the guidewire system of the present invention, the outer wire shown in the advanced position and the intermediate stiffener wire in the retracted position;

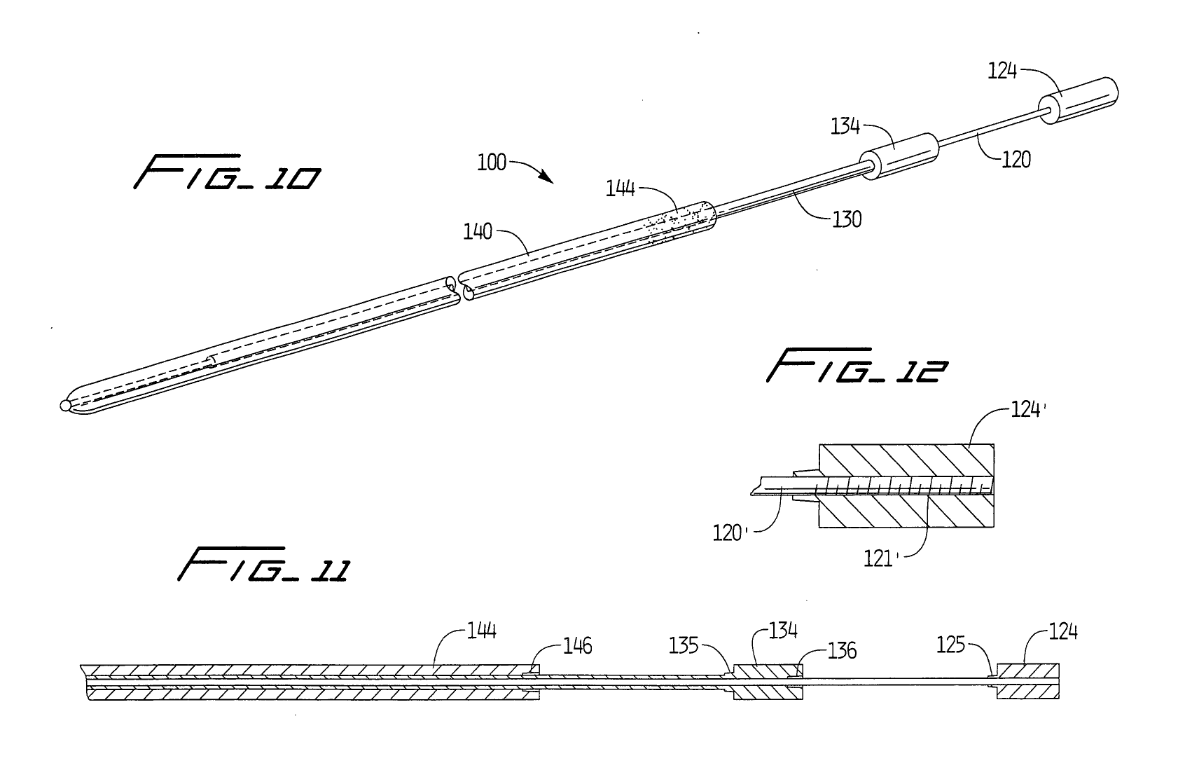

FIG. 11 is a longitudinal cross-sectional view of the guidewire of FIG. 10 showing the outer wire in the advanced position and the intermediate stiffener wire in the retracted position;

FIG. 12 is a cross-sectional view of an alternate embodiment of the handle of the inner wire having a threaded engagement for removal from the inner wire;

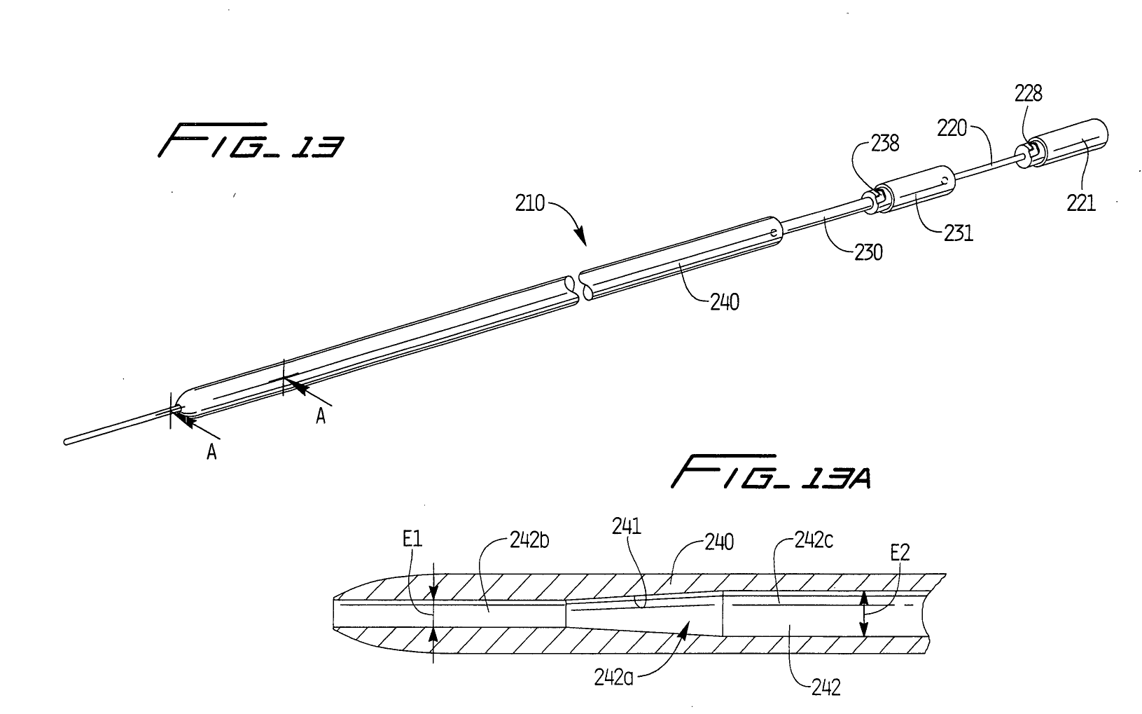

FIG. 13 is a perspective view of an alternate embodiment of the guidewire system of the present invention showing the intermediate (stiffener) tube and outer tube in the retracted position to expose the inner wire;

FIG. 13A is a cross-sectional view taken along line A-A of FIG. 13 showing the distal region of the outer tube (the inner wire removed for clarity);

FIG. 13B is an exploded perspective view of the guidewire of FIG. 13;

FIG. 14 is an enlarged view of the guidewire of FIG. 13 showing the handles in the retracted unlocked position;

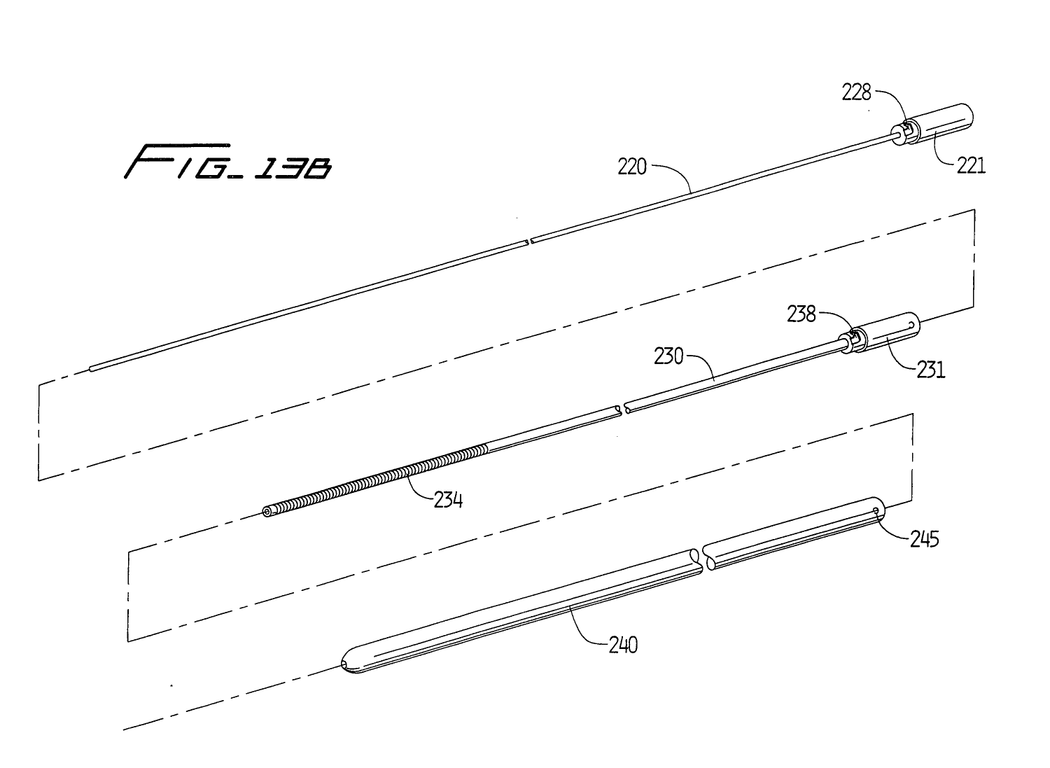

FIG. 15 is a perspective view of the inner wire handle of FIG. 14 engaged (interlocked) with the stiffener handle prior to locking;

FIG. 16 is a perspective view similar to FIG. 15 showing the inner wire handle rotated to lock the inner wire and stiffener;

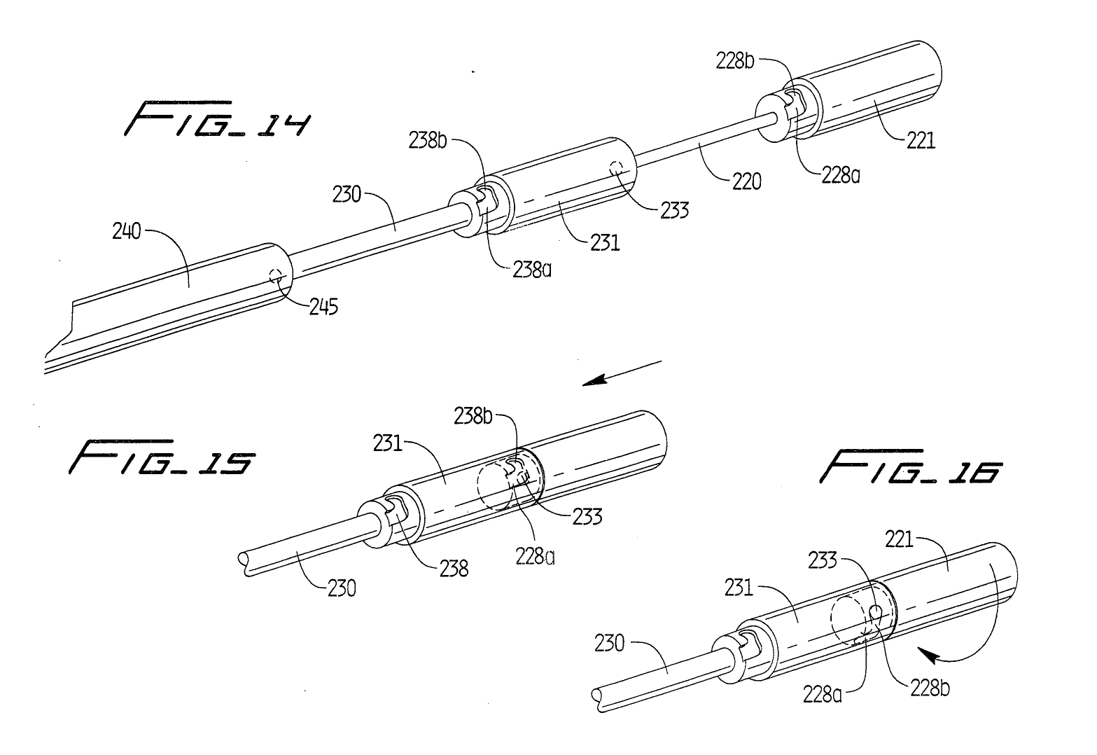

FIG. 17 is an enlarged view of the stiffener tube of FIG. 13;

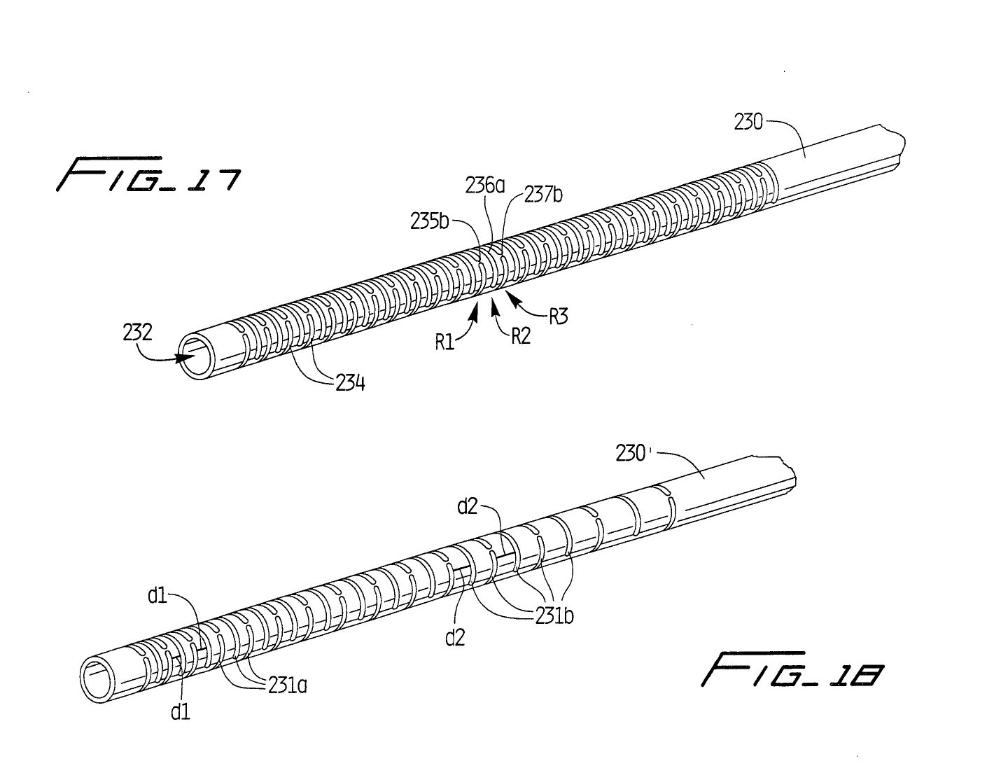

FIG. 18 is an enlarged view of an alternate embodiment of the stiffener tube;

FIG. 18A is an enlarged view of an alternate embodiment of the outer tube;

FIG. 19 is an enlarged perspective view of a proximal portion of an alternate embodiment of the guidewire system of the present invention showing the inner wire and stiffener tube in the retracted position;

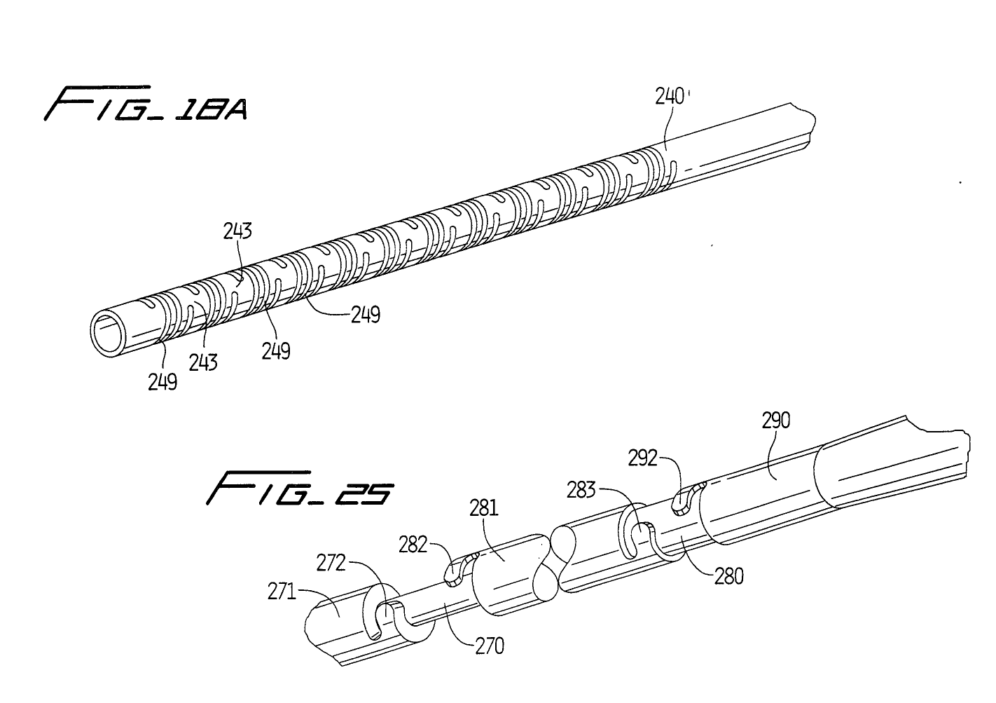

FIG. 20 is a perspective view showing the handles of FIG. 19 prior to engagement;

FIG. 21 is a cross-sectional view of the handle of the inner wire of FIG. 19 prior to attachment to the inner wire;

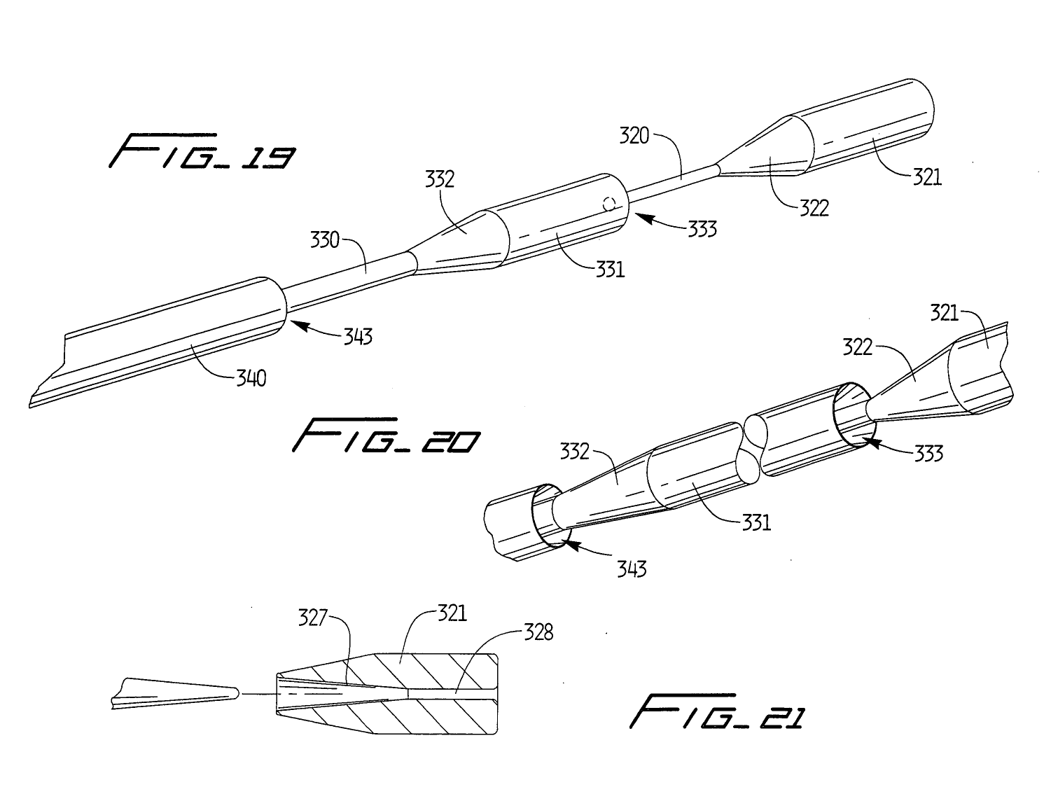

FIG. 22 is an enlarged perspective view of a proximal portion of another alternate embodiment of the guidewire system of the present invention showing the inner wire and stiffener in the retracted position;

FIG. 22A is an enlarged view of the locking member of the inner wire of FIG. 22;

FIG. 23 is a cross-sectional view illustrating the threaded locking member of the inner wire spaced from the threaded portion of the stiffener collar;

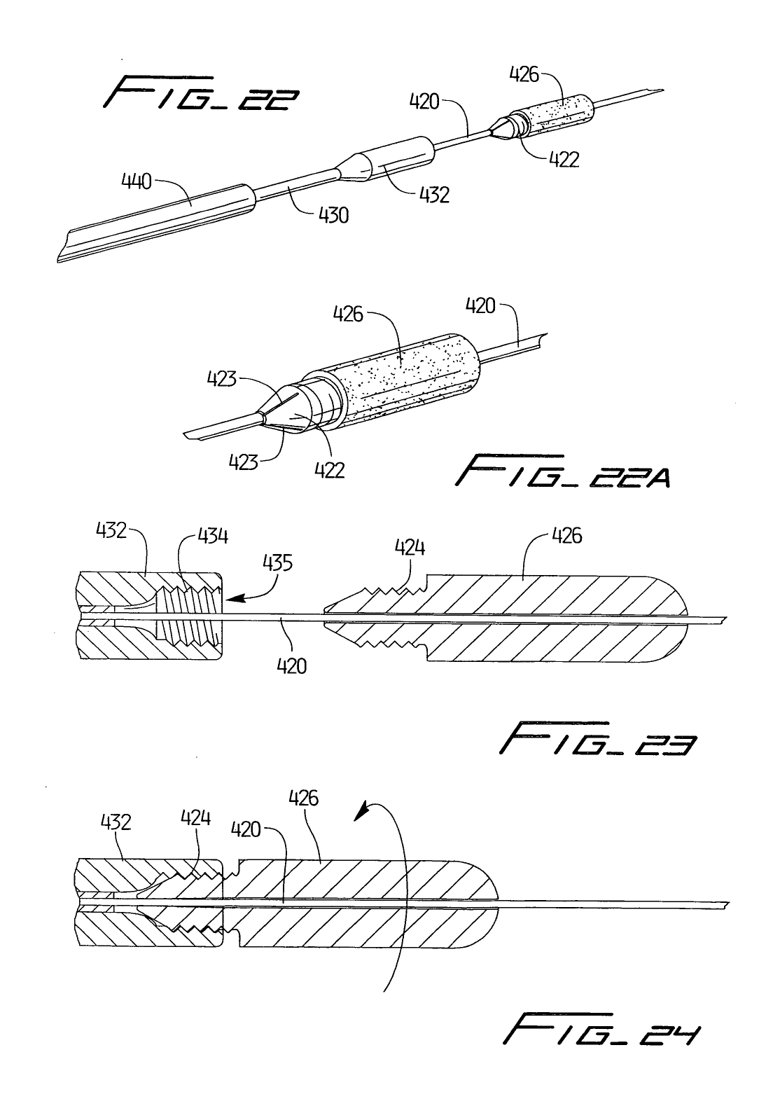

FIG. 24 is a cross-sectional view of the threaded locking members engaged prior to further rotation to fix the inner wire axially with respect to the stiffener tube; and

FIG. 25 is a perspective view of a proximal portion of another alternate embodiment of the guidewire system of the present invention showing the inner wire in the retracted position.

### DETAILED DESCRIPTION OF PREFERRED EMBODIMENTS

Turning now to the drawings, wherein like reference numerals identify similar or like components throughout the several views, the guidewire system of the present invention is illustrated. The guidewire system comprises a guidewire 10 have three coaxial members movable with respect to one another to adjust the stiffness and size (outer diameter) of the guidewire.

More specifically, the guidewire system 10 in the embodiment shown in FIGS. 1-5, comprises a small diameter inner member 20, an intermediate stiffener member 30 slidable over the inner member 20, and a larger diameter outer member 40 slidable over the intermediate member 30 and the inner member 20. As used herein, the term “proximal” refers to closer to the user and the term “distal” refers to further from the user. The term member as used herein includes a wire, tube or other structure of the inner, intermediate and outer components of the guidewire system.

The small diameter inner member 20, in a first embodiment, is a wire having a spherical or ball tip 22 either integral or attached thereto. The ball tip 22 provides a blunt atraumatic leading end of the wire to reduce trauma to the vessel during advancement. The ball tip 22 is also preferably dimensioned so it has a larger diameter (transverse dimension) than the diameter of the lumen 42 of the outer wire 40 or at least larger than the diameter of the opening to the lumen 42. Thus, it also acts as a stop to prevent withdrawal of the entire wire 20 through the outer wire 40 and acts as a stop to limit distal movement of the outer wire 40 so it does not extend over the tip 24 so that a blunt tip can remain as the leading edge for the guidewire 10 to provide a smoother passage. This is shown for example in FIG. 2 where the surface 22a of the tip 22 would abut the distalmost end 40a of outer wire 40.

It should be appreciated that tips other than ball tips can be utilized. For example, FIGS. 6 and 7 show a conical tip 22′ of inner wire 20′ having a smother transition and functioning similar to ball tip 22. In all other respects, guidewire 10′ of FIG. 6 is identical to the guidewire 10 of FIG. 1. The guidewire 10′ is shown in FIG. 6 with the intermediate wire 30′ and outer wire 40′ retracted to expose the inner wire 22′ and shown in FIG. 7 with the outer wire 40′ advanced to its distal position.

Additionally, it should be appreciated that an enlarged tip need not be provided. For example, in the alternate embodiment of FIG. 2, the distal tip of the inner wire is the same diameter as the portion proximal of the distal tip.

The inner wire forms the core wire of the system, and is preferably formed of a solid core and is preferably composed at least in part of a shape memory material such as Nitinol. Non-metallic materials can also be utilized, such as Pebax. The inner wire in one embodiment can have a coil and core combination towards its distal end and is a solid wire towards it proximal end. Other materials such as stainless steel are also contemplated. Preferably the wire 20 has an outer diameter of about 0.014 inches, although other dimensions are also contemplated. Preferably, the inner wire 20 has a greater degree of flexibility and is softer than the other two wires 30, 40.

The stiffener member 30 forms the intermediate wire as it is positioned between the inner wire 20 and outer wire 40. Stiffener wire 30 can be formed from single or multiple wires wound together, having a lumen 32 with a dimension (diameter) larger than the outer diameter of the wire 20 so it can slide over wire 20 (or wire 20 can side within it). In a preferred embodiment, the stiffener wire 30 has an outer diameter of about 0.018 inches, although other dimensions are also contemplated. The wire 20 is preferably formed of a shape memory material such as Nitinol, although other materials, such as stainless steel, are also contemplated. In one embodiment, the stiffener has a stiffness/rigidity greater than the stiffness of the inner wire 20 and outer wire 40. However, the stiffener can alternatively have a stiffness less than the stiffness of the outer wire/and or inner wire, provided it has sufficient stiffness such that when it is advanced, it stiffens a distal region of the outer wire (and overall guidewire system) by providing a distal region of increased wall thickness due to the combination of stiffener and outer member. That is, in such embodiment, advancement of the stiffener provides a thicker walled and thereby stiffer/more rigid wire.

The stiffener, in an alternate embodiment, is in the form of a slotted hypotube which can be as described in more detail below.

The outer wire 40 has a longitudinally extending opening or lumen 42 with a dimension (diameter) larger than the outer diameter of the intermediate wire 30 so it can slide over wire 30 and smaller wire 20 (or wire 30 can slide within it). In a preferred embodiment, the outer diameter of the wire is between about 0.035 inches to about 0.038 inches, although other dimensions are also contemplated. In one embodiment (not shown) the outer wire 40 is a wound wire wound in one direction. It could be a round wire or a rectangular wire. Alternatively, it can comprise a series of wound or twisted wires. The wire 40 can also have a hydrophilic and/or a PTFE coating. It can also be formed with a coated or uncoated plastic jacket. A safety wire connected to proximal and distal portions of the outer wire could optionally be provided. The outer wire 40 has a stiffness/rigidity greater than the stiffness of the inner wire 20. In some embodiments, the outer wire can also have a stiffness less than the stiffness/rigidity of the intermediate wire 30 as discussed above.

In an alternate embodiment, the outer tube is in the form of a slotted hypotube which can be as described in more detail below.

In the alternate embodiment of FIGS. 2 and 2A, inner wire 50 does not have an enlarged tip but terminates in a tip 52 of the same diameter. Outer wire 60 has a thicker wall portion at the distal end portion 62 to create a shoulder 62b and a reduced lumen diameter 62a. The shoulder 62b can form a stop to limit distal advancement of the stiffener 70 such that the distalmost end of the stiffener, although extending to a distal region of the outer wire 60, cannot extend to a distalmost end of the outer wire 60. The reduced lumen area 62a creates a tighter fit for the inner wire 20 as it slides more closely around the inner wire 50 to limit entry of material into the lumen of the outer wire 60. The tighter fit also enables clot to be wiped off the inner wire 50 upon movement with respect to the distal tip 63 of outer wire 60. The tip 63 also has a smooth shallow taper (similar to the outer wire 40 of FIG. 1) to provide a smoother transition and facilitate advancement over the inner wire 50 in very tight and tortuous anatomy with reduced trauma. Tips with even more gradual tapers could be provided. In all other respects, the guidewire system of FIG. 2 is the same as FIG. 1.

In one embodiment, the inner wires described herein have a length of about 3.0 m, the intermediate wires or tubes described herein have a length of about 2.36 m to about 2.38 m and the outer wires or tubes described herein have a length of about 2.4 m to about 2.6 m. It should be understood that these dimensions are provided by way of example and other dimensions are also contemplated.

It should be appreciated that sliding movement of the wires (or tubes) referred to herein means that either the outside wire (or tube) is moving over the held (stationary) inside wire, the inside wire is moving within the stationary outside wire, or both wires are sliding in opposite directions. For example, the inner wire can be exposed by moving the inner wire distally, moving the outer wire proximally, or moving both wires in their respective directions. However, it may be preferable that the stiffening wire be advanced or retracted to maintain the advanced position of the guidewire during insertion. The foregoing likewise applies to the use of tubes instead of wires as one or more of the members of the guidewire system.

The use of the guidewire system will now be described with reference to the embodiment of FIG. 1, it being understood that such use is also applicable to the other embodiments of the present invention described herein utilizing the three members in the form of wires or tubes (or other structures).

In use, selective positioning of the wires with respect to one another varies the diameter of the guidewire being advanced through the vascular system and varies the stiffness of the guidewire. This independent sliding movement of the wires provides an in situ progressive transformation of the soft wire, used to avoid damage to the vessel, into a stiff or rigid wire to provide a rail system for easier catheter advancement thereover and to increase pushability around curved anatomy.

More specifically, to increase the pushability and stiffness of the guidewire 10, the outer wire 40 is advanced distally over the inner wire 20 from the position of FIG. 4 to the position of FIG. 5 (or the inner wire 20 is retracted to the position of FIG. 5). If further stiffness or enhanced pushability is desired, the intermediate wire 30 is advanced from the retracted position of FIG. 5 to the advanced position of FIG. 1B. Sliding of the wires is controlled by the user at the proximal end.

Note in the embodiment of FIG. 1B, in the advanced position of the intermediate wire 30, it remains spaced proximally from the distalmost end of the outer wire 40 to reduce trauma to the vessel by ensuring some flexibility of the distalmost tip of the guidewire 10. In one embodiment, in the advanced position, the distalmost end 34 of the intermediate wire 30 is spaced a distance of about 2-4 centimeters from the distalmost end 40a of outer wire 40. Other spaced distances are also contemplated. In the advanced position of the inner wire 20 (FIGS. 1 and 4), it preferably protrudes about 30 cm to about 40 cm from the distalmost end 40a of outer wire 40. Other protruding lengths are also contemplated.

After the guidewire 10 has been stiffened by relative sliding movement of the outer and/or intermediate wire, if a smaller diameter and more flexible guidewire is desired, the inner wire 20 can again be exposed by retraction of the outer wire 40 (and stiffener wire 30) or advancement of the inner wire 20 (or opposite movement of both).

As can be appreciated, relative movement of the wires can occur repeatedly as desired to enhance advancement of the guidewire 10 though the vascular system to the desired surgical site.

In an alternate embodiment shown in FIGS. 10 and 11, each of the wires 120, 130 and 140 of guidewire 100 has a handle portion. Handle portions as used herein include integral handles, separate handles attached to the members or a proximal end portion of the member which interlocks with another member. With reference to FIGS. 10 and 11, inner wire 120 has a handle 124 at its proximal end, intermediate stiffener wire 130 has a handle 134 at its proximal end, and outer wire 140 has a handle portion 144 at its proximal end. This facilitates grasping of the wire by the user as well as facilitates torquing of the wire to rotate the distal end. One or more of the handles can include a textured surface (see e.g. handle 144 of FIG. 10) to facilitate gripping.

The handles can optionally interlock to fix the positioning of the wires with respect to one another. FIG. 11 illustrates one way to interlock the handles. In this embodiment, the engagement regions of the members include an interlocking feature in the form of a taper/recess interlock. More specifically, interlocking is achieved by providing a taper on the distal portion of handles 124 and 134 which frictionally mate with a proximal recess at the proximal end of the mating handle. More specifically, distal tapered region 125 of handle 124 would frictionally engage with the proximal recess 136 of handle 134 and distal tapered region 135 of handle 134 would frictionally engage the proximal recess 146 of handle 144. Thus, when inner wire 120 is moved relative to the outer wire 140, the user does not need to hold it in this advanced (exposed) position as the handle 124 would interlock with handle 134 to fix the inner wire 120 in position. Similarly, when intermediate wire 130 is moved relative to the outer wire 140, the user does not need to hold it in this position as the handle 134 would interlock with handle 144 to fix the inner wire 120 in position. This interlocking of the handles 134 and 144 could also be used to maintain the spacing between the distalmost ends of the wires 130 and 140 as described above with respect to wires 30 and 40. It could also be used to maintain the distal tip of the inner wire 20 as the leading edge instead of or in addition to utilizing the larger diameter tip, e.g. the ball tip, to achieve this function. The handle for the outer wire is shown as the same dimension of the outer wire so the handle can be considered the proximal portion of the wire.

FIGS. 13-17 illustrate an alternate embodiment of the guidewire system having alternate engagement regions providing an alternate mechanism for interlocking the members. This system also has a stiffener and outer member formed of a tube. The relative stiffness of the inner, intermediate, and outer members is provided as discussed above.

More specifically, guidewire 210 has an inner member 220, an intermediate stiffening member 230 and an outer member 240. Stiffener member 230 is in the form of a tube, preferably composed of stainless steel, and has a longitudinally extending lumen 232 (FIG. 17) dimensioned to slidingly receive inner wire 220. The stiffener tube 230 in the embodiment illustrated in FIG. 17 has a plurality of slots 234 formed therein (preferably laser cut into the tube) to increase the flexibility of the tube. Each slot in the illustrated embodiment, extends around a portion of the circumference, for less than 360 degrees and preferably less than 180 degrees. Additionally, the slots are staggered such that a solid portion of the tube between the space between slots in one row is adjacent a slotted portion of another row. For ease of understanding, three rows of slots have been numbered in FIG. 17 to illustrate how slot portion 236a of row R2 is adjacent a gap 235b (solid tube portion) between slot portions of row R1 and adjacent gap 237b (solid tube portion) between slot portions of row R3.

As shown, the axial spacing between the slots in FIG. 17 is substantially equal. However, it is also contemplated that the spacing between the slots can be varied at various portions along the tube to provide areas of different flexibility. For example, in the embodiment of FIG. 18, the slots of tube 230′ vary such that slots 231a at the distal portion of the tube 230′ are closer together (have a shorter distance d1) than the slots 231b of a more proximal portion which have a greater distance d2 between them. This provides more flexibility toward the distal end. Various slot spacing is contemplated. For example, the slots can be varied such that they become progressively further apart in a proximal direction or discrete regions of the tube can have slots of substantially equal spacing, but different than other regions of the tube.

It is also contemplated, that the slots can be formed in a spiral pattern such as shown in FIG. 18A illustrating an outer tube with slots. The outer tube 240′ has spiral or helically arranged slots 249 formed in the tube, preferably at an angle to the longitudinal axis as shown. The spiral slots, preferably formed by laser cutting, can be interrupted, leaving a solid wall portion 243 between the sets of spiraling slots. The solid wall portions can be evenly spaced as shown to provide similar sets of slots or can be varied to provide sets having different lengths of spiraling slots. Such spiraling slots can also be formed on the intermediate stiffener tube. A heat shrink tube (not shown), made of PET for example, can be positioned over all or a portion of the tube.

It should be appreciated that in an alternate embodiment, the stiffener tube and/or outer tube do not have slots.

Referring back to FIGS. 13-13C, inner wire preferably is a 0.014″ wire as described above and outer member 240 is in the form of a tube, preferably of stainless steel. The outer tube 240 can have slots in the various arrangements as described above with respect to the stiffener tube 230 and the distances between slots can be varied in different regions of the tube as described above. The outer tube 240 and stiffener 230 can have the same or different slot arrangements.

Outer tube 240 has a lumen 242 dimensioned to slidingly receive stiffener tube 230. Outer tube 240 has a distal end portion, best shown in FIG. 13A, having a distal lumen portion 242a that gradually reduces in diameter, to a diameter E1 at region 242b, less than the diameter E2 at region 242c. In this manner, diameter E1 can be close to the outer diameter of the inner wire 230 to reduce any gap between the inner wire 220 and outer tube 240 when the inner wire 220 is extended. The inner wall 241 of outer tube 240 is angled to provide a smooth transition between the two diameters E1 and E2 to ease the movement of inner wire 220 through lumen 242 to an extended position.

The members in the embodiment of FIGS. 13-16 have engagement regions with an interlocking feature in the form of a rotational pin and slot arrangement. More specifically, inner wire 220 has a handle 221 with an L-shaped slot 228 at its distal end. Pin 233 at the proximal end of handle 231 of stiffener tube 220 engages slot 228. That is, when the inner wire 220 is advanced longitudinally, the pin 233 engages the longitudinal region 228a of slot 228 (see FIG. 15). This also acts as a stop for longitudinal advancement of the inner wire 220. Once in the slot region 228a, the inner wire 220 is rotated so that the pin 233 enters the transverse slot region 228b as shown in FIG. 16, thereby fixing the axial position of the inner wire 220 and stiffener 230. Similarly, the intermediate tube 230 has an L-shaped slot 238 at the distal end of handle 231. A proximal pin 245 of outer tube 240 enters the longitudinal slot region 238a and then upon rotation, enters the transverse region 238b to fix the stiffener 230 to the outer tube 240. Pin 245 could also be provided on a handle of outer tube 240. This interlocking handle also functions as a stop to limit the extent of distal movement of the stiffener tube 230 within outer tube 240.

Note as an alternative to the pin/slot arrangement, two locking tabs could be provided as shown in FIG. 25. Mating tabs 292 and 283 of outer tube 290 and of handle 281 of stiffener tube 280, respectively, interlock upon rotation. Similarly, proximal locking tab 282 of handle 281 of stiffener tube 280 interlocks with tab 272 of handle 271 of inner wire 270.

FIGS. 19-21 illustrate another embodiment for interlocking the handles to lock the members to prevent longitudinal movement of the members. The embodiment is similar to the embodiment of FIG. 11. Inner wire 320 has a proximal handle 321 with a distal tapered region 322. This tapered region 322 is inserted into the opening 333 of proximal handle 331 of stiffener tube 330 to frictionally engage the handles. This interference fit interlocks the handles which thereby interlocks the inner wire 320 and stiffener tube 330 to prevent movement of the inner wire 320 with respect to the stiffener tube. The proximal end of outer tube 340 has an opening 343 dimensioned to matingly receive the distal tapered region 332 of handle 331 of intermediate stiffener tube 330 to lock the stiffener 330 against longitudinal movement with respect to the outer tube 340.

The handle 321 of inner wire 320 can include a distal taper 327 to releasably engage the inner wire 320, as shown in FIG. 21. In this manner, the handle 321 can be removed from the wire 320 to enable removal of the intermediate tube 330 and outer tube 340 from the surgical site. The proximal end of the handle 321 can include a lumen 328 to engage an extension wire (not shown) to increase the length of the inner wire 320.

Alternately, a torque type handle can be used to control the inner wire and can be positioned at a desired portion along the proximal exposed wire and can be envisioned to be configured so as to lock and unlock on the other wires while at the same time engaging the handle of the other wire. FIGS. 22-24 illustrate an example of this showing another alternate embodiment of an engagement region with and interlocking feature. A collet 422 has a distal tapered region with a plurality of slots 423. A series of external threads 424 threadingly engage internal threads 434 of collar 432. Collar 432 is attached to a proximal end of the stiffener tube 430.

In use, collet 422, which encircles inner wire 420, is inserted within the opening 435 of handle or collar 434. In this position, collet 422 is attached to collar 434 but inner wire 420 can still freely move longitudinally within intermediate stiffener tube 430 and outer tube 440. If the user decides to fix (lock) the position of the inner wire 420 to prevent longitudinal movement, handle surface 426, preferably textured to enhance grasping, is gripped and rotated as shown in FIG. 24. This advances the collet 422 further into the collar 432, resulting in the internal taper of the collar compressing the slotted region of the collet 422 to apply a clamping force on the inner wire 420. This clamping force applied by the collet 424 prevents longitudinal movement of the inner wire 420. To free the inner wire 420 for longitudinal movement, the collet 424 is rotated in the opposite direction to retract the collet 424 to allow it to expand to loosen the grip on the inner wire 420.

In an alternate embodiment shown in FIG. 12, the inner wire handle 124′ is removable from inner wire 120′ by unscrewing. More specifically, handle 124′ is attached to inner wire 120′ by a screw thread 121′ such that the handle 124′ can be unscrewed from inner wire 120. This allows outer wire 140 and intermediate wire 130 to be removed by retraction (proximal movement) over the length of the inner wire 120′, thereby leaving only the softer, smaller diameter wire in place.

A conventional extension wire W can optionally be attached to the inner wire 20 (or other inner wires described herein) by a friction fit as shown in FIGS. 8 and 9. That is, a recessed portion of female taper of inner wire 20 receives a male tapered distal end W1 of extension wire W.

It is also contemplated that the outer and intermediate wires could be held in place and the inner wire removed and replaced with another 0.014 wire, such as a conventional 0.014 wire currently being used for surgical procedures.

The aforedescribed guidewires of the present invention provide a method of adjusting the stiffness and size of a guidewire without full withdrawal of the guidewire from a patient's vascular system. The use will be described in conjunction with guidewire 10, however it should be appreciated that the description is applicable to the other guidewires discussed herein.

In one method of use, the guidewire 10 is advanced into the vascular system from a remote site, such as the femoral artery F (see FIG. 3), with the outer wire 40 and stiffener 30 in the retracted position to expose a substantial length of the inner wire 10 to expose a smaller wire diameter as shown in FIGS. 1 and 4. This provides for increased flexibility of the guidewire system and less trauma to the vessel. Note it is also contemplated that the guidewire is inserted from other sites such as the jugular vein or radial artery.

After initial advancement of the guidewire 10 through the vascular system en route to the target site such as the carotid artery C (FIG. 3), if a tortuous vessel portion or other anatomy is encountered wherein the inner wire 20 lacks the requisite pushability and stiffness, the outer wire 40 is slid in a distal direction over the inner wire 20 without removing the inner wire 20 from the patient. This creates a stiffer guidewire to increase the pushability of the guidewire system 10 to enable it to advance through the curved vessel portion (see FIG. 5)

If during advancement, the outer wire 40 lacks the requisite pushability or stiffness to advance through a tortuous vessel portion or other anatomy, the stiffener 30 can be advanced in a distal direction within the outer wire 40 and over the inner wire 20 to increase the overall stiffness of the guidewire 10, as shown in FIG. 1B.

After advancing through the tortuous vessel, the stiffener 30 can be withdrawn if desired, leaving the more flexible outer wire 40 for advancement.

If during advancement of the guidewire 10 with outer wire 40 covering the inner wire 20 a restricted passage in the portion of the vessel is encountered such that the vessel lumen dimension is less than the outer diameter of the outer wire 40, the outer wire 40 can be retracted in a proximal direction to expose a substantial length of the inner wire 20. The smaller diameter inner wire 20 can then be used to advance through the restricted passage of the vessel lumen.

As can be appreciated, the wires can be slid relative to one another (as defined herein) during the advancement of guidewire 10 to the treatment site any number of times as desired to provide the requisite diameter size, flexibility and stiffness.

Once the treatment site is reached, the stiffener 30 and outer wire 40 can be slid proximally over the inner wire 20 and removed from the patient, thereby leaving the inner wire 20 in the patient to function as a rail for over the wire catheter insertion. Alternatively, the guidewire 10 can remain in place with the larger diameter wire 40 functioning as a rail for over the wire catheter insertion.

Although the method of use was described in relation to guidewire 10, the other guidewires disclosed herein would be advanced in a similar fashion. In the embodiment with a handle, the handle or torquer would be removed if it was desired to remove the outer wire and stiffener.

Additionally, the method was described above with the guidewire system initially inserted so the inner wire extends from the outer wire. It is also contemplated that if a larger wire is desired for initial insertion, the guidewire system would be inserted with the inner wire retracted. Then the inner wire can be advanced to be exposed if a smaller size or increased pushability is desired.

While the above description contains many specifics, those specifics should not be construed as limitations on the scope of the disclosure, but merely as exemplifications of preferred embodiments thereof. For example, one or more of the wires can contain a hydrophilic coating. Those skilled in the art will envision many other possible variations that are within the scope and spirit of the disclosure as defined by the claims appended hereto.

1-21. (canceled)

22. A medical guidewire system comprising a core wire, a first tube and a second tube, the first tube having a second diameter larger than a first diameter of the core wire and including a distal region having a greater flexibility than a flexibility of a proximal region, the second tube having a third diameter larger than the second diameter, the second tube having a distal region having a greater flexibility than a flexibility of a proximal region, the first tube having a longitudinally extending opening to receive the core wire for relative sliding movement, the second tube having a longitudinally extending opening to receive the first tube for relative sliding movement, the first tube configured to increase the stiffness of a distal end of the second tube.

23. The guidewire system of claim 22, wherein the core wire includes a solid core for at least a majority of its length.

24. The guidewire system of claim 22, wherein the first tube has a slotted region in at least the distal region.

25. The guidewire system of claim 22, wherein the second tube has a slotted region in at least the distal region.

26. The guidewire system of claim 24, wherein distal slots of the first tube are closer together than more proximal slots.

27. The guidewire system of claim 25, wherein distal slots of the second tube are closer together than more proximal slots.

28. The guidewire system of claim 25, wherein the second tube has a plurality of interrupted slots.

29. The guidewire system of claim 24, wherein the first tube has a plurality of interrupted slots.

30. The guidewire system of claim 22, wherein the first tube is positionable in the second tube in a retracted position and a fully advanced position, in the fully advanced position, a distalmost tip of the first tube does not extend distal of a distalmost tip of the second tube.

31. The guidewire system of claim 22, wherein the core wire has a handle configured and dimensioned to receive a catheter thereover.

32. A medical guidewire system comprising:
an inner member having a first diameter and a first stiffness;
a stiffener member having a second diameter larger than the first diameter and having a second stiffness greater than the first stiffness; and
an outer member having a third diameter larger than the second diameter and having a third stiffness greater than the first stiffness and less than the second stiffness, the stiffener member being slidable over the inner member and slidable within the outer member to increase the stiffness of the outer member.

33. The guidewire system of claim 32, wherein the outer member comprises a tube with a least one slot in a distal region to provide a more flexible distal region.

34. The guidewire system of claim 33, wherein the stiffener member comprises a tube with a least one slot in a distal region to provide a more flexible distal region.

35. The guidewire system of claim 32, wherein the stiffener member comprises a tube with a least one slot in a distal region to provide a more flexible region.

36. A medical guidewire system comprising:
an inner member having a first diameter and a first stiffness;
a stiffener member having a proximal end and a distal end and a second diameter larger than the first diameter, the stiffener having a second stiffness greater than the first stiffness; and
an outer member having a third diameter larger than the second diameter and having a third stiffness greater than the first stiffness, the outer member having a proximal end and a distal end, the stiffener being positioned within the outer member for relative sliding movement for positioning in a proximal position with respect to the outer member and a fully advanced distal position with respect to the outer member, wherein in the distal position a distalmost tip of the stiffener member does not extend distal of a distalmost tip of the outer member.

37. The guidewire system of claim 36, wherein the outer member includes an internal shoulder, the shoulder creating a stop to limit distal advancement of the stiffener member.

38. The guidewire system of claim 36, wherein the outer member has a lumen, the lumen having a reduced diameter area in a distal area to ease the movement of the inner member to an advanced position where a distalmost tip of the inner member is positioned distal of a distalmost tip of the outer member.

39. The guidewire system of claim 36, wherein the inner member is advanceable to a position wherein a distalmost tip of the inner member is positioned distally of the distalmost tip of the outer member.

40. The guidewire system of claim 36, wherein a distal end of the outer member tapers to a transverse dimension close to the first diameter of the inner member

41. A medical guidewire system comprising:
an inner member having a first diameter and a first stiffness;
a stiffener member having a second diameter larger than the first diameter and having a second stiffness greater than the first stiffness; and
an outer member having a third diameter larger than the second diameter, the outer member having a lumen dimensioned to receive the stiffener member, a distal portion of the lumen having a first transverse dimension less than a second transverse dimension of a more proximal portion to reduce a gap between the inner member and outer member when the inner member is advanced so a distalmost tip extends distally of a distalmost tip of the outer member, the first transverse dimension being less than the second diameter of the stiffener member.

42. The guidewire system of claim 41, wherein the distalmost tip of the outer member is tapered.

43. The guidewire system of claim 41, wherein an inner wall of the lumen is angled inwardly to transition from the second transverse dimension to the first transverse dimension.

44. The guidewire system of claim 41, wherein the second stiffness of the stiffener member is greater than a third stiffness of the outer member.

45. The guidewire system of claim 41, wherein the inner and outer members comprise tubes with slotted regions.

46. A medical guidewire system comprising:
an inner member having a first diameter and a first stiffness, the inner member being dimensioned for passage of a catheter thereover;
an intermediate member having a second handle, a second diameter larger than the first diameter and having a second stiffness greater than the first stiffness; and
an outer member having a third handle, a third diameter larger than the second diameter and a third stiffness greater than the first stiffness, the outer member having a lumen dimensioned to receive the stiffener, wherein the first, second and third handles are dimensioned to receive a catheter thereover.

47. The guidewire system of claim 46, wherein the inner member has a first handle, the first handle being cylindrically shaped.

48. The guidewire system of claim 47, wherein the second and third handles are cylindrically shaped.

49. The guidewire system of claim 46, wherein the second and third handles are cylindrically shaped.

50. The guidewire system of claim 45, wherein the first and second handles interlock.

51. The guidewire system of claim 50, wherein the second handle includes a collar to compress a collet on the inner member to fix the position of the inner member and intermediate member.

52. A medical guidewire system comprising:
an inner member having a first diameter and a first stiffness;
a stiffener member having a second diameter larger than the first diameter and having a second stiffness greater than the first stiffness, the stiffener member further including a clamping member to apply a clamping force to a portion of the inner member to fix the position of the inner member with respect to the stiffener member; and
an outer member having a third diameter larger than the second diameter and having a third stiffness greater than the first stiffness, the outer member having a lumen dimensioned to receive the stiffener member.

53. The guidewire system of claim 52, wherein the clamping member comprises a collar and the portion of the inner member comprises a collet, the collar applying a clamping force to the collet

54. The guidewire system of claim 52, wherein the lumen of the outer member has a reduced diameter in a distal region.

55. The guidewire system of claim 52, wherein the clamping member is dimensioned to receive a catheter thereover.

56. The guidewire system of claim 52, wherein the clamping member is configured to fix the position of the inner member at a number of selected positions of the inner member with respect to the stiffener member.

57. A medical guidewire system having a transverse dimension and comprising an inner member, an intermediate member and an outer member, the guidewire having first, second and third guidewire stiffnesses, wherein the first guidewire stiffness is achieved by advancement of a distalmost tip of the inner member beyond a distalmost tip of the outer member, the second guidewire stiffness is achieved by the inner member and intermediate member being positioned in a retracted position with respect to the outer member, and the third guidewire stiffness is achieved by advancing the intermediate member and without changing the transverse dimension of the guidewire, the third guidewire stiffness being greater than the second guidewire stiffness.

58. The guidewire system of claim 57, wherein the inner member includes a handle at a proximal region, the handle dimensioned to receive a catheter thereover.

59. The guidewire system of claim 57, wherein the intermediate member includes a handle at a proximal region, the handle dimensioned to receive a catheter thereover.

60. The guidewire system of claim 57, wherein in the advanced position of the intermediate member, a distalmost tip of the intermediate member does not extend distal of a distalmost tip of the outer member.

61. The guidewire system of claim 57, wherein the intermediate and outer members comprises a hypotube with a series of slots formed in a sidewall to increase flexibility.

62. The guidewire system of claim 61, wherein the space between the slots increases toward a proximal end of the hypotube.

63. A guidewire system having an inner member having a first outer diameter and a first stiffness, an outer member having a third diameter larger than the first diameter, and a stiffener member positioned between the inner and outer members and having a second outer diameter larger than the first outer diameter and smaller than the third diameter and having a second stiffness greater than the first stiffness, wherein the inner member and stiffener member are movable relative to one another and the inner member and stiffener member are selectively lockable to fix a position of the inner member relative to the stiffener member in a position proximal to a fully advanced position with respect to the outer member.

64. The guidewire system of claim 63, wherein the inner member and stiffener member are selectively by selectively applying a clamping force on an outer surface portion of the inner member.
